(19) United States
(12) Patent Application Publication
(10) Pub. No.: US 2011/0014189 A1
(43) Pub. Date: Jan. 20, 2011

(54) Stable pharmaceutical composition comprising at least one monoclonal antibody and at least one amphiphilic polysaccharide comprising hydrophobic substituents

(75) Inventors: Olivier Soula, Meyzieu (FR); Gerard Soula, Meyzieu (FR); Martin Gaudier, Lyon (FR); Remi Soula, Lyon (FR)

Correspondence Address: OLIFF & BERRIDGE, PLC, P.O. BOX 320850, ALEXANDRIA, VA, 22320-4850, US

(73) Assignee: ADOCIA, LYON (FR)

(21) Appl. No.: 12/654,552

(22) Filed: Dec. 23, 2009

Related U.S. Application Data
(60) Provisional application No. 61/193,789, filed on Dec. 23, 2008.

(30) Foreign Application Priority Data
Dec. 23, 2008 (FR) 08.07438

(51) Int. Cl. A61K 39/395; A61P 17/00; A61P 35/00; A61P 11/00; A61P 29/00; A61P 37/00; A61P 9/00; A61P 25/00; A61P 1/00; A61P 3/00; A61P 5/00

(52) U.S. Cl. 424/133.1; 424/141.1; 424/142.1

(57) ABSTRACT

A stable pharmaceutical composition with at least one monoclonal antibody and at least one amphiphilic polysaccharide chosen from the group of amphiphilic polysaccharides comprising carboxylate functional groups partly substituted with at least one hydrophobic substituent is disclosed.

Monoclonal antibodies have in recent years met with phenomenal success due to their exceptional efficacy in treating certain cancers and a certain number of chronic diseases affecting a large number of patients. Among these diseases, mention may be made of various forms of cancer, prostate cancer, breast cancer, liver cancer, but also other pathologies such as rheumatoid arthritis, certain infectious diseases, age-related macular degeneration, etc.

A few compounds of this family are already reference medicaments for these pathologies.

As the therapeutic value of monoclonal antibodies is established, many biopharmaceutical companies have engaged in the development of novel compounds, which may have superior therapeutic effects while at the same time having lesser side effects.

However, these monoclonal antibodies must, for the most part, be administered in large amount in order to achieve the desired therapeutic effect.

One major difficulty consists in obtaining pharmaceutical compositions containing the required amount of protein, with a sufficient storage stability to ensure its efficacy over time and to avoid the formation of by-products that might have side effects, in particular immunogenic effects.

Specifically, it is observed that these monoclonal antibodies, which are high molecular weight proteins, readily aggregate under the effect of temperature or a mechanical stress. This is observed even in products such as Avastin and Erbitux, which are currently marketed. They need to be filtered before use, so as to remove the particles that have precipitated. It is obvious that, under these conditions, the amount of active material administered and the nature and amount of the impurities that are not filtered out cannot be controlled.

Many attempts have been made to obtain stable pharmaceutical compositions of monoclonal antibodies in high concentrations.

Examples that will be mentioned include:
patent application NZ534542 in the name of Chugai, which relates to stable formulations of anti-interleukin 6 or anti-HM1.24 receptor antibody, which contain a sugar as stabilizer, said sugar being a disaccharide or trisaccharide nonreducing sugar;
patent application WO 2006/044 908 in the name of Genentech, which describes stable formulations of monoclonal antibodies in a histidine buffer, said formulations possibly comprising, inter alia, disaccharides, especially trehalose and sucrose;
patent application WO 2008/121 615 in the name of Medimune, which relates to anti-interferon antibody formulations, said formulations comprising, inter alia, a buffer of histidine citrate buffer type, etc., but also trehalose or sucrose.

A large proportion of the work conducted is limited to finding, for a given antibody, a buffer that is effective for conserving the biological activity. The solutions provided on a case-by-case basis therefore cannot be generalized and, what is more, often prove to be ineffective, as may be observed for many commercial products.

The present invention makes it possible to overcome the problem of stability of monoclonal antibodies by using polysaccharides simultaneously comprising carboxylate groups and hydrophobic substituents.

In particular, the Applicant has demonstrated that said modified polysaccharides simultaneously comprising carboxylate and hydrophobic groups:
stabilize antibodies with respect to aggregation and precipitation,
increase the solubility,
aid dissolution.

The present invention generally makes it possible to solve the problems of stability of monoclonal antibodies. It concerns a stable pharmaceutical composition comprising at least one monoclonal antibody and at least one amphiphilic polysaccharide.

For example, a stable composition will be a composition comprising a monoclonal antibody and an amphiphilic polysaccharide in which no aggregation is detected after incubation for 48 hours at 56° C., in aqueous solution at the working concentration.

In one embodiment, the amphiphilic polysaccharide is chosen from polysaccharides comprising carboxyl functional groups, at least one of which is substituted with at least one hydrophobic radical, noted Hy:
said hydrophobic radical (Hy) being grafted or bound to the anionic polysaccharide either:
via a function F′, said function F′ resulting from coupling between a reactive function of a hydrophobic compound and a carboxyl function of the anionic polysaccharide,
via a linker R, said linker R being linked to the polysaccharide via a bond F resulting from coupling between a reactive function of the precursor of the linker R′ and a carboxyl function of the anionic polysaccharide and said hydrophobic radical (Hy) being linked to the linker R via a function G resulting from coupling between a reactive function of a hydrophobic compound and a reactive function of the precursor of the linker R′;
the carboxyl functions of the unsubstituted anionic polysaccharide being in the form of the carboxylate of a cation, preferably an alkali metal cation such as Na⁺ or K⁺;
F being either an amide, ester, thioester or anhydride function,
F′ being either an amide, ester, thioester or anhydride function,
G being either an amide, ester, thioester, thionoester, carbamate, carbonate or anhydride function,
Hy being a radical resulting either from coupling between a reactive function of a hydrophobic compound and a carboxyl function of the anionic polysaccharide, or from coupling between a reactive function of a hydrophobic compound and a reactive function of the precursor of the linker R′, consisting of a chain comprising between 4 and 50 carbons, optionally branched and/or unsaturated, optionally comprising one or more heteroatoms, such as O, N and/or S, optionally comprising one or more saturated, unsaturated or aromatic rings or heterocycles,
R being a divalent radical consisting of a chain comprising between 1 and 18 carbons, optionally branched and/or unsaturated, optionally comprising one or more heteroatoms, such as O, N and/or S, optionally comprising one or more saturated, unsaturated or aromatic rings or heterocycles and resulting from the reaction of a precursor R′ containing at least two identical or different reactive functions chosen from the group consisting of alcohol, acid, amine, thiol and thio acid functions,
said polysaccharide comprising carboxyl functional groups being amphiphilic at neutral pH.

In one embodiment, the polysaccharides comprising carboxyl functional groups are polysaccharides naturally bearing carboxyl functional groups and are chosen from the group consisting of alginate, hyaluronan and galacturonan.

In one embodiment, the polysaccharides comprising carboxyl functional groups are synthetic polysaccharides obtained from polysaccharides naturally comprising carboxyl functional groups or from neutral polysaccharides, on which at least 15 carboxyl functional groups per 100 saccharide units have been grafted, of general formula I:

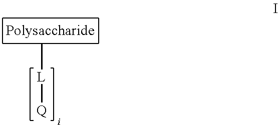

the natural polysaccharides being chosen from the group of polysaccharides predominantly consisting of glycoside monomers linked via glycoside bonds of (1,6) and/or (1,4) and/or (1,3) and/or (1,2) type,
L being a bond resulting from coupling between the linker Q and an —OH function of the polysaccharide and being either an ester, thionoester, carbonate, carbamate or ether function,
i represents the mole fraction of the substituents L-Q per saccharide unit of the polysaccharide,
Q being a chain comprising between 1 and 18 carbons, optionally branched and/or unsaturated, comprising one or more heteroatoms, such as O, N and/or S, and comprising at least one carboxyl functional group, —CO₂H.

In one embodiment, the polysaccharide is consisted predominantly of glycoside monomers linked via glycoside bonds of (1,6) type.

In one embodiment, the polysaccharide consisted predominantly of glycoside monomers linked via glycoside bonds of (1,6) type is dextran.

In one embodiment, the polysaccharide is consisted predominantly of glycoside monomers linked via glycoside bonds of (1,4) type.

In one embodiment, the polysaccharide consisted predominantly of glycoside monomers linked via glycoside bonds of (1,4) type is chosen from the group consisting of pullulan, alginate, hyaluronan, xylan, galacturonan or a water-soluble cellulose.

In one embodiment, the polysaccharide is a pullulan.

In one embodiment, the polysaccharide is an alginate.

In one embodiment, the polysaccharide is a hyaluronan.

In one embodiment, the polysaccharide is a xylan.

In one embodiment, the polysaccharide is a galacturonan.

In one embodiment, the polysaccharide is a water-soluble cellulose.

In one embodiment, the polysaccharide is consisted predominantly of glycoside monomers linked via glycoside bonds of (1,3) type.

In one embodiment, the polysaccharide consisted predominantly of glycoside monomers linked via glycoside bonds of (1,3) type is a curdlan.

In one embodiment, the polysaccharide is consisted predominantly of glycoside monomers linked via glycoside bonds of (1,2) type.

In one embodiment, the polysaccharide consisted predominantly of glycoside monomers linked via glycoside bonds of (1,2) type is an inulin.

In one embodiment, the polysaccharide is consisted predominantly of glycoside monomers linked via glycoside bonds of (1,4) and (1,3) type.

In one embodiment, the polysaccharide formed predominantly from glycoside monomers linked via glycoside bonds of (1,4) and (1,3) type is a glucan.

In one embodiment, the polysaccharide is consisted predominantly of glycoside monomers linked via glycoside bonds of (1,4), (1,3) and (1,2) type.

In one embodiment, the polysaccharide consisted predominantly of glycoside monomers linked via glycoside bonds of (1,4), (1,3) and (1,2) type is mannan.

In one embodiment, the polysaccharide according to the invention is characterized in that the group Q is chosen from the following groups:

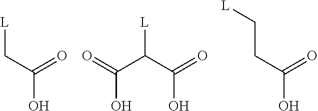

In one embodiment, i is between 0.1 and 3.

In one embodiment, i is between 0.2 and 1.5.

In one embodiment, the polysaccharides are polysaccharides comprising carboxyl functional groups, at least one of which is substituted with a hydrophobic alcohol derivative, noted Ah:
said hydrophobic alcohol (Ah) being grafted or linked to the anionic polysaccharide via a coupling arm R, said coupling arm being linked to the anionic polysaccharide via a function F, said function F resulting from coupling between an amine, alcohol, thioalcohol or carboxyl function of the precursor of the linker R′ and a carboxyl function of the anionic polysaccharide, and said coupling arm R being linked to the hydrophobic alcohol via a function G resulting from coupling between a carboxyl, amine, thio acid or alcohol function of the precursor of the coupling arm R′ and an alcohol function of the hydrophobic alcohol, the carboxyl functions of the unsubstituted anionic polysaccharide being in the form of a carboxylate of a cation, preferably of an alkali metal cation, such as Na⁺ or K⁺;
   F being either an amide function or an ester function, or a thioester function, or an anhydride function,
   G being either an ester function, or a thioester function, or a carbonate function, or a carbamate function,
   R being a divalent radical consisting of a chain comprising between 1 and 18 carbons, optionally branched and/or unsaturated, optionally comprising one or more heteroatoms, such as O, N and/or S,
   Ah being a hydrophobic alcohol or thioalcohol residue, produced from coupling between the hydroxyl function of the hydrophobic alcohol and at least one reactive function borne by the precursor of the divalent radical R,
said polysaccharide comprising carboxyl functional groups being amphiphilic at neutral pH.

In one embodiment, F is an amide function, G is an ester function, R′ is an amino acid and Ah is a hydrophobic alcohol residue.

In one embodiment, F is an amide function, G is a thioester function, R′ is an amino acid and Ah is a hydrophobic thioalcohol residue.

In one embodiment, F is an amide function, G is a carbamate function, R′ is a diamine and Ah is a hydrophobic alcohol residue.

In one embodiment, F is an amide function, G is a carbonate function, R′ is an amino alcohol and Ah is a hydrophobic alcohol residue.

In one embodiment, F is an amide function, G is a thionoester function, R′ is an 0-thioamino acid and Ah is a hydrophobic alcohol residue.

In one embodiment, F is an ester function, G is an ester function, R′ is an acid alcohol and Ah is a hydrophobic alcohol residue.

In one embodiment, F is an ester function, G is a thioester function, R′ is an acid alcohol and Ah is a hydrophobic thioalcohol residue.

In one embodiment, F is an ester function, G is a carbonate function, R′ is a dialcohol and Ah is a hydrophobic alcohol residue.

In one embodiment, F is an ester function, G is a carbamate function, R′ is an alcoholamine and Ah is a hydrophobic alcohol residue.

In one embodiment, F is a thioester function, G is an ester function, R′ is an acid thiol and Ah is a hydrophobic alcohol residue.

In one embodiment, F is a thioester function, G is a thioester function, R′ is an acid thiol and Ah is a hydrophobic thioalcohol residue.

In one embodiment, F is a thioester function, G is a carbonate function, R′ is an alcohol thiol and Ah is a hydrophobic alcohol residue.

In one embodiment, F is a thioester function, G is a carbamate function, R′ is an aminethiol and Ah is a hydrophobic alcohol residue.

In one embodiment, F is an anhydride function, G is an ester function, R′ is a diacid and Ah is a hydrophobic alcohol residue.

In one embodiment, F is an anhydride function, G is a thioester function, R′ is a diacid and Ah is a hydrophobic thioalcohol residue.

In one embodiment, F is an anhydride function, G is a carbamate function, R′ is an amino acid and Ah is a hydrophobic alcohol residue.

In one embodiment, F is an anhydride function, G is a carbonate function, R′ is an acid alcohol and Ah is a hydrophobic alcohol residue.

In one embodiment, said polysaccharide comprising carboxyl functional groups partly substituted with hydrophobic alcohols is chosen from polysaccharides comprising carboxyl functional groups of general formula II:

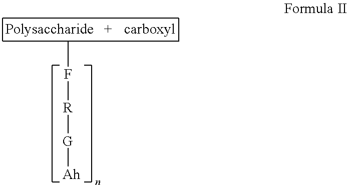

in which n represents the mole fraction of the carboxyl functions of the polysaccharide that are substituted with F-R-G-Ah and is between 0.01 and 0.7,
F, R, G and Ah correspond to the definitions given above, and when the carboxyl function of the polysaccharide is not substituted with F-R-G-Ah, then the carboxyl functional group(s) of the polysaccharide are carboxylates of an alkali metal cation, preferably such as Na⁺ or K⁺.

In one embodiment, the precursor of the group R, R′ is characterized in that it is chosen from amino acids.

In one embodiment, the amino acids are chosen from α-amino acids.

In one embodiment, the α-amino acids are chosen from natural α-amino acids.

In one embodiment, the natural α-amino acids are chosen from leucine, alanine, isoleucine, glycine, phenylalanine, tryptophan, valine and proline.

In one embodiment, the precursor of the group R, R′ is characterized in that it is chosen from polyols.

In one embodiment, the polyols are chosen from dialcohols.

In one embodiment, the dialcohols are chosen from the group consisting of diethylene glycol and triethylene glycol:

In one embodiment, the dialcohols are chosen from the group consisting of polyethylene glycols without any mass restriction.

In one embodiment, the polyols are chosen from the group consisting of glycerol, diglycerol and triglycerol.

In one embodiment, the polyol is triethanolamine.

In one embodiment, the precursor of the group R, R′ is characterized in that it is chosen from diamines.

In one embodiment, the diamines are chosen from the group consisting of ethylenediamine and lysine and derivatives thereof.

In one embodiment, the precursor of the group R, R′ is characterized in that it is chosen from alcohol amines.

In one embodiment, the alcohol amines are chosen from the group consisting of ethanolamine, 2-aminopropanol, isopropanolamine, 3-amino-1,2-propanediol, diethanolamine, diisopropanolamine, tromethamine (Tris) and 2-(2-aminoethoxy)ethanol.

In one embodiment, the alcohol amines are chosen from the group consisting of reduced amino acids.

In one embodiment, the reduced amino acids are chosen from the group consisting of alaminol, valinol, leucinol, isoleucinol, prolinol and phenylalaminol.

In one embodiment, the alcohol amines are chosen from the group consisting of charged amino acids.

In one embodiment, the charged amino acids are chosen from the group consisting of serine and threonine.

In one embodiment, the precursor of the group R, R′ is characterized in that it is chosen from diacids.

In one embodiment, the diacid is chosen from the group consisting of succinic acid, glutamic acid, maleic acid, oxalic acid, malonic acid, fumaric acid and glutaconic acid.

In one embodiment, the precursor of the group R, R′ is characterized in that it is chosen from alcohol acids.

In one embodiment, the alcohol acids are chosen from the group consisting of mandelic acid, lactic acid and citric acid.

In one embodiment, the hydrophobic alcohol is chosen from fatty alcohols.

In one embodiment, the hydrophobic alcohol is chosen from alcohols consisting of a saturated or unsaturated, branched or unbranched alkyl chain comprising from 4 to 18 carbons.

In one embodiment, the hydrophobic alcohol is chosen from alcohols consisting of a saturated or unsaturated, branched or unbranched alkyl chain comprising from 6 to 18 carbons.

In one embodiment, the hydrophobic alcohol is chosen from alcohols consisting of a saturated or unsaturated, branched or unbranched alkyl chain comprising more than 18 carbons.

In one embodiment, the hydrophobic alcohol is chosen from alcohols formed from a saturated or unsaturated, branched or unbranched alkyl chain comprising more than 18 carbons.

In one embodiment, the hydrophobic alcohol is octanol.

In one embodiment, the hydrophobic alcohol is dodecanol.

In one embodiment, the hydrophobic alcohol is 2-ethylbutanol.

In one embodiment, the fatty alcohol is chosen from myristyl alcohol, cetyl alcohol, stearyl alcohol, cetearyl alcohol, butyl alcohol, oleyl alcohol and lanolin.

In one embodiment, the hydrophobic alcohol is chosen from cholesterol derivatives.

In one embodiment, the cholesterol derivative is cholesterol.

In one embodiment, the hydrophobic alcohol is chosen from menthol derivatives.

In one embodiment, the hydrophobic alcohol is menthol in, its racemic form.

In one embodiment, the hydrophobic alcohol is the D isomer of menthol.

In one embodiment, the hydrophobic alcohol is the L isomer of menthol.

In one embodiment, the hydrophobic alcohol is chosen from tocopherols.

In one embodiment, the tocopherol is α-tocopherol.

In one embodiment, the α-tocopherol is racemic α-tocopherol.

In one embodiment, the tocopherol is the D isomer of α-tocopherol.

In one embodiment, the tocopherol is the L isomer of α-tocopherol.

In one embodiment, the hydrophobic alcohol is chosen from alcohols bearing an aryl group.

In one embodiment, the alcohol bearing an aryl group is chosen from benzyl alcohol and phenethyl alcohol.

In one embodiment, the hydrophobic alcohol is chosen from unsaturated fatty alcohols in the group consisting of geraniol, (3-citronellol and farnesol.

In one embodiment, the hydrophobic alcohol is 3,7-dimethyl-1-octanol.

In one embodiment, the polysaccharides are polysaccharides comprising carboxyl functional groups, at least one of said carboxyl groups being substituted with a hydrophobic alcohol derivative, noted Ah:
said hydrophobic alcohol (Ah) being grafted or linked to the anionic polysaccharide via a function F′, said function. F′ resulting from coupling between the carboxylate function of the anionic polysaccharide and the hydroxyl function of the hydrophobic alcohol, the unsubstituted carboxyl functions of the anionic polysaccharide being in the form of the carboxylate of a cation, preferably of an alkali metal cation such as Na⁺ or K⁺;
   F′ being an ester or thioester function,
Ah being a hydrophobic alcohol residue or a hydrophobic thioalcohol residue,
said polysaccharide comprising carboxyl functional groups being amphiphilic at neutral pH.

In one embodiment, the polysaccharide comprising carboxyl functional groups partly substituted with hydrophobic alcohols is chosen from polysaccharides comprising carboxyl functional groups of general formula III:

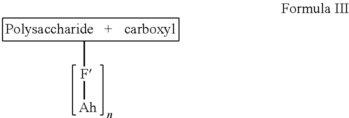

in which n represents the mole fraction of carboxyl functions of the polysaccharide substituted with -F′-Ah and is between 0.01 and 0.7,
F′ and Ah correspond to the definitions given above, and when the carboxyl function of the polysaccharide is not substituted with F′-Ah, then the carboxyl functional group(s) of the polysaccharide are carboxylates of a cation, preferably of an alkali metal cation such as Na⁺ or K⁺.

In one embodiment, the hydrophobic alcohol is chosen from fatty alcohols.

In one embodiment, the hydrophobic alcohol is chosen from alcohols consisting of a saturated or unsaturated, branched or unbranched alkyl chain comprising from 6 to 18 carbons.

In one embodiment, the hydrophobic alcohol is chosen from alcohols consisting of a saturated or unsaturated, branched or unbranched alkyl chain comprising from 8 to 18 carbons.

In one embodiment, the hydrophobic alcohol is chosen from alcohols consisting of a saturated or unsaturated, branched or unbranched alkyl chain comprising more than 18 carbons.

In one embodiment, the hydrophobic alcohol is octanol.

In one embodiment, the hydrophobic alcohol is 2-ethylbutanol.

In one embodiment, the hydrophobic alcohol is dodecanol.

In one embodiment, the fatty alcohol is chosen from myristyl alcohol, cetyl alcohol, stearyl alcohol, cetearyl alcohol, butyl alcohol, oleyl alcohol and lanolin.

In one embodiment, the hydrophobic alcohol is chosen from cholesterol derivatives.

In one embodiment, the cholesterol derivative is cholesterol.

In one embodiment, the hydrophobic alcohol is chosen from menthol derivatives.

In one embodiment, the hydrophobic alcohol is menthol in its racemic form.

In one embodiment, the hydrophobic alcohol is the D isomer of menthol.

In one embodiment, the hydrophobic alcohol is the L isomer of menthol.

In one embodiment, the hydrophobic alcohol is chosen from tocopherols.

In one embodiment, the tocopherol is α-tocopherol.

In one embodiment, the α-tocopherol is racemic α-tocopherol.

In one embodiment, the tocopherol is the D isomer of α-tocopherol.

In one embodiment, the tocopherol is the L isomer of α-tocopherol.

In one embodiment, the hydrophobic alcohol is chosen from alcohols bearing an aryl group.

In one embodiment, the alcohol bearing an aryl group is chosen from benzyl alcohol and phenethyl alcohol.

In one embodiment, the hydrophobic alcohol is chosen from unsaturated fatty alcohols in the group consisting of geraniol, β-citronellol and farnesol.

In one embodiment, the hydrophobic alcohol is 3,7-dimethyl-1-octanol.

In one embodiment, the amphiphilic polysaccharides are polysaccharides comprising carboxyl functional groups, at least one of which is substituted with a hydrophobic amine derivative, noted Amh:
said hydrophobic amine being grafted or linked to the anionic polysaccharide via an amide function F′, said amide function F′ resulting from coupling between the amine function of the hydrophobic amine and a carboxyl function of the anionic polysaccharide, the unsubstituted carboxyl functions of the anionic polysaccharide being in the form of a carboxylate of a cation, preferably of an alkali metal cation such as Na+ or K+,
Amh being a hydrophobic amine residue produced by coupling between the amine function of the hydrophobic amine and a carboxyl function of the anionic polysaccharide.

In one embodiment, the polysaccharide comprising carboxyl functional groups grafted with hydrophobic amines is chosen from polysaccharides comprising carboxyl functional groups of general formula IV:

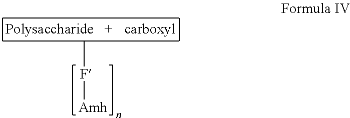

in which n represents the mole fraction of carboxyl, functions of the polysaccharide that are substituted with F′-Amh and is between 0.01 and 0.7,
F′ and Amh satisfying the definitions given above, and when the carboxyl function of the polysaccharide is not substituted with F′-Amh, then the carboxyl function(s) of the polysaccharide are carboxylates of a cation, preferably of an alkali metal cation such as Na+ or K+.

In one embodiment, the hydrophobic amine is chosen from amines consisting of a saturated or unsaturated, branched or linear alkyl chain comprising from 6 to 18 carbons.

In one embodiment, the fatty amine is dodecylamine.

In one embodiment, the fatty amine is chosen from myristylamine, cetylamine, stearylamine, cetearylamine, butylamine, oleylamine and lanolin.

In one embodiment, the hydrophobic amine is chosen from amines bearing an aryl group.

In one embodiment, the amine bearing an aryl group is chosen from benzylamine and phenethylamine.

In one embodiment, the polysaccharides are polysaccharides comprising carboxyl functional groups, at least one of said groups being substituted with a hydrophobic acid derivative, noted Ach:
said hydrophobic acid (Ach) being grafted or linked to the anionic polysaccharide via an anhydride function F′, said function F resulting from coupling between the carboxyl function of the anionic polysaccharide and the carboxyl function of the hydrophobic acid, the unsubstituted carboxyl functions of the anionic polysaccharide being in the form of the carboxylate of a cation, preferably of an alkali metal cation such as Na⁺ or K⁺,
Ach being a hydrophobic acid or hydrophobic O-thioacid residue,
said polysaccharide comprising carboxyl functional groups being amphiphilic at neutral pH.

In one embodiment, the polysaccharide comprising carboxyl functional groups partly substituted with hydrophobic acids is chosen from polysaccharides comprising carboxyl functional groups of general formula V:

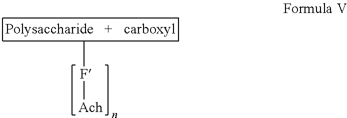

in which n represents the mole fraction of the carboxyl functions of the polysaccharide, that are substituted with -F′-Ach and is between 0.01 and 0.7,
F′ and Ach corresponding to the definitions given above, and when the carboxyl function of the polysaccharide is not substituted with F′-Ach, then the carboxyl functional group(s) of the polysaccharide are carboxylates of a cation, preferably of an alkali metal cation such as Na⁺ or K⁺.

In one embodiment, the hydrophobic acid is chosen from fatty acids.

In one embodiment, the fatty acids are chosen from the group consisting of acids consisting of a saturated or unsaturated, branched or unbranched alkyl chain comprising from 6 to 50 carbons.

In one embodiment, the fatty acids are chosen from the group consisting of linear fatty acids.

In one embodiment, the linear fatty acids are chosen from the group consisting of caproic acid, enanthic acid, caprylic acid, capric acid, nonanoic acid, decanoic acid, undecanoic acid, dodecanoic acid, palmitic acid, stearic acid, arachidic acid, behenic acid, tricosanoic acid, lignoceric acid, heptacosanoic acid, octacosanoic acid and melissic acid.

In one embodiment, the fatty acids are chosen from the group consisting of unsaturated fatty acids.

In one embodiment, the unsaturated fatty acids are chosen from the group consisting of myristoleic acid, palmitoleic acid, oleic acid, elaidic acid, linoleic acid, α-linoleic acid, arachidonic acid, eicosapentaenoic acid, erucic acid and docosahexaenoic acid.

In one embodiment, the fatty acids are chosen from the group consisting of bile acids and derivatives thereof.

In one embodiment, the bile acids and derivatives thereof are chosen from the group consisting of cholic acid, dehydrocholic acid, deoxycholic acid and chenodeoxycholic acid.

In one embodiment, the polysaccharides are polysaccharides comprising carboxyl functional groups, at least one of which is substituted with a hydrophobic acid derivative, noted Ach:
said hydrophobic acid (Ach) being grafted or linked to the anionic polysaccharide via a coupling arm R, said coupling arm being linked to the anionic polysaccharide via a function F, said function F resulting from coupling between an amine, alcohol, thioalcohol or carboxyl function of the precursor of the linker R′ and a carboxyl function of the anionic polysaccharide, and said coupling arm R being linked to the hydrophobic acid via a function G resulting from coupling between an amine, alcohol, thioalcohol or carboxyl function of the precursor of the coupling arm R′ and a carboxyl function of the hydrophobic acid, the unsubstituted carboxyl functions of the anionic polysaccharide being in the form of the carboxylate of a cation, preferably of an alkali metal cation such as Na⁺ or K⁺,
   F being either an amide function, or an ester function, or a thioester function, or an anhydride function,
   G being either an ester function, or an amide function, or an ester function, or a thioester function, or an anhydride function,
   R being a divalent radical consisting of a chain comprising between 1 and 18 carbons, optionally branched and/or unsaturated, optionally comprising one or more heteroatoms such as O, N and/or S,
   Ach being a residue of an acid, produced by coupling between the carboxyl function of the hydrophobic acid and at least one reactive function borne by the precursor R′ of the divalent radical R,
said polysaccharide comprising carboxyl functional groups being amphiphilic at neutral pH.

In one embodiment, F is an amide function, G is an ester function, R′ is an alcoholamine and Ach is a hydrophobic acid residue.

In one embodiment, F is an amide function, G is a thioester function, R′ is a thiolamine and Ach is a hydrophobic acid residue.

In one embodiment, F is an amide function, G is an amide function, R′ is a diamine and Ach is a hydrophobic acid residue.

In one embodiment, F is an amide function, G is an anhydride function, R′ is an amino acid and Ach is a hydrophobic acid residue.

In one embodiment, F is an ester function, G is an amide function, R′ is an alcoholamine and Ach is a hydrophobic acid residue.

In one embodiment, F is an ester function, G is an ester function, R′ is a dialcohol and Ach is a hydrophobic acid residue.

In one embodiment, F is an ester function, G is a thioester function, R′ is an alcoholthiol and Ach is a hydrophobic acid residue.

In one embodiment, F is an ester function, G is an anhydride function, R′ is an acid alcohol and Ach is a hydrophobic acid residue.

In one embodiment, F is a thioester function, G is an amide function, R′ is a thiolamine and Ach is a hydrophobic acid residue.

In one embodiment, F is a thioester function, G is an ester function, R′ is an alcoholthiol and Ach is a hydrophobic acid residue.

In one embodiment, F is a thioester function, G is a thioester function, R′ is a dithioalcohol and Ach is a hydrophobic acid residue.

In one embodiment, F is a thioester function, G is an anhydride function, R′ is a thiol acid and Ach is a hydrophobic acid residue.

In one embodiment, F is an anhydride function, G is an ester function, R′ is an alcohol acid and Ach is a hydrophobic acid residue.

In one embodiment, F is an anhydride function, G is a thioester function, R′ is a thiol acid and Ach is a hydrophobic acid residue.

In one embodiment, F is an anhydride function, G is an amide function, R′ is an amino acid and Ach is a hydrophobic acid residue.

In one embodiment, F is an anhydride function, G is an anhydride function, R′ is a diacid and Ach is a hydrophobic acid residue.

In one embodiment, said polysaccharide comprising carboxyl functional groups partly substituted with hydrophobic alcohols is chosen from polysaccharides comprising carboxyl functional groups of general formula VI:

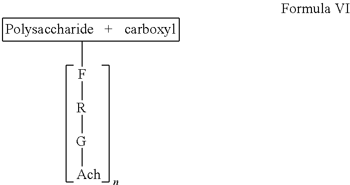

in which n represents the mole fraction of carboxyl functions of the polysaccharide that are substituted with F-R-G-Ach and is between 0.01 and 0.7,
F, R, G and Ach correspond to the definitions given above, and when the carboxyl function of the polysaccharide is not substituted with F-R-G-Ach, then the carboxyl functional group(s) of the polysaccharide are carboxylates of a cation, preferably an alkali metal cation such as Na⁺ or K⁺.

In one embodiment, the precursor of the group R, R′ is characterized in that it is chosen from amino acids.

In one embodiment, the amino acids are chosen from α-amino acids.

In one embodiment, the α-amino acids are chosen from natural α-amino acids.

In one embodiment, the natural α-amino acids are chosen from leucine, alanine, isoleucine, glycine, phenylalanine, tryptophan, valine and proline.

In one embodiment, the hydrophobic alcohol is chosen from fatty alcohols.

In one embodiment, the precursor of the group R, R′ is characterized in that it is chosen from dialcohols.

In one embodiment, the dialcohols are chosen from the group formed by glycerol, diglycerol and triglycerol.

In one embodiment, the dialcohol is triethanolamine.

In one embodiment, the dialcohols are chosen from the group consisting of diethylene glycol and triethylene glycol.

In one embodiment, the dialcohols are chosen from the group consisting of polyethylene glycols, without mass restriction.

In one embodiment, the precursor of the group R, R′ is characterized in that it is chosen from diamines.

In one embodiment, the diamines are chosen from the group consisting of ethylenediamine and lysine and derivatives thereof.

In one embodiment, the precursor of the group R, R′ is characterized in that it is chosen from alcoholamines.

In one embodiment, the alcoholamines are chosen from the group consisting of ethanolamine, 2-aminopropanol, isopropanolamine, 3-amino-1,2-propanediol, diethanolamine, diisopropanolamine, tromethamine (Tris) and 2-(2-aminoethoxy)ethanol.

In one embodiment, the alcoholamines are chosen from the group consisting of reduced amino acids.

In one embodiment, the reduced amino acids are chosen from the group consisting of alaminol, valinol, leucinol, isoleucinol, prolinol and phenylalaminol.

In one embodiment, the alcoholamines are chosen from the group consisting of charged amino acids.

In one embodiment, the charged amino acids are chosen from the group consisting of serine and threonine.

In one embodiment, the precursor of the group R, R′ is characterized in that it is chosen from diacids.

In one embodiment, the diacid is chosen from the group consisting of succinic acid, glutamic acid, maleic acid, oxalic acid, malonic acid, fumaric acid and glutaconic acid.

In one embodiment, the precursor of the group R, R′ is characterized in that it is chosen from alcohol acids.

In one embodiment, the alcohol acids are chosen from the group consisting of mandelic acid, lactic acid and citric acid.

In one embodiment, the hydrophobic acid is chosen from fatty acids.

In one embodiment, the fatty acids are chosen from the group consisting of acids consisting of a saturated or unsaturated, branched or unbranched alkyl chain comprising from 6 to 50 carbons.

In one embodiment, the fatty acids are chosen from the group consisting of linear fatty acids.

Caproic acid, enanthic acid, caprylic acid, capric acid, nonanoic acid, decanoic acid, undecanoic acid, dodecanoic acid, palmitic acid, stearic acid, arachidic acid, behenic acid, tricosanoic acid, lignoceric acid, heptacosanoic acid, octacosanoic acid and melissic acid.

In one embodiment, the fatty acids are chosen from the group formed by unsaturated fatty acids.

In one embodiment, the unsaturated fatty acids are chosen from the group consisting of myristoleic acid, palmitoleic acid, oleic acid, elaidic acid, linoleic acid, a-linoleic acid, arachidonic acid, eicosapentaenoic acid, erucic acid and docosahexaenoic acid.

In one embodiment, the fatty acids are chosen from the group formed by bile acids and derivatives thereof.

In one embodiment, the bile acids and derivatives thereof are chosen from the group consisting of cholic acid, dehydrocholic acid, deoxycholic acid and chenodeoxycholic acid.

The polysaccharide may have a degree of polymerization m of between 10 and 10 000.

In one embodiment, the polysaccharide has a degree of polymerization m of between 10 and 1000.

In another embodiment, the polysaccharide has a degree of polymerization m of between 10 and 500.

In one embodiment, the invention relates to a composition characterized in that the antibody is chosen from the group of therapeutically active antibodies and fragments thereof.

In one embodiment, the antibodies or fragments thereof are chosen from the group of antibodies or antibody fragments used in cancerology, targeting:

CD 52, VEGF (vascular endothelial growth factor), EGF-R (epidermal growth factor receptor), CD 11a, CCR4 (chemokine C—C receptor 4), CD 105, CD 123, CD 137, CD 19, CD 22, CD 23, CD 3, CD 30, CD 38, CD 4, CD 40, CD 55SC-1, CD 56, CD 6, CD 74, CD 80, CS1 (cell-surface glycoprotein 1), CTLA4 (cytotoxic T-lymphocyte antigen 4, also known as CD152), DR5 (death receptor 5), Ep-CAM (epithelial cell adhesion molecule), folate receptor alpha, ganglioside GD2, ganglioside GD3, GPNMB, glycoprotein NMB, HGF/SF (hepatocyte growth factor/scatter factor), IGF-1 (insulin-like growth factor), IGF1-receptor (insulin-like growth factor-1 receptor), IL 13 (interleukin-13), IL 6 (interleukin-6), IL-6R (interleukin-6 receptor), immunodominant fungal antigen heat shock protein 90 (hsp90), integrin alpha 5 beta 3, MHC (major histocompatibility complex) class II, MN-antigen (also known as G250-antigen), MUC1, PD-1 (programmed death 1), PIGF (placental growth factor), PDGFRa (platelet-derived growth factor receptor alpha), prostate specific membrane antigen (PSMA), PTHrP (parathyroid hormone-related protein), CD200 receptor, receptor activator of nuclear factor kappa B ligand (RANKL), sphingosine-1-phosphate (S1P), TGF beta, (transforming growth factor beta), TRAIL (tumor necrosis factor (TNF)-related apoptosis-inducing ligand) receptor 1, tumor necrosis factor receptor 2, vascular endothelial growth factor receptor 2 (VEGFR-2), CD 33, CD 20 or CA125 (cancer antigen 125).

In one embodiment, the antibodies are chosen from the group of antibodies comprising alemtuzumab, bevacizumab, cetuximab, efalizumab, gemtuzumab, britumomab, ovarex mab, panitumumab, rituximab, tositumomab or trastuzumab.

In one embodiment, the antibodies are chosen from the group of antibodies or antibody fragments used in dermatology, targeting:

TNF alpha (tumor necrosis factor alpha), IL 12, IL 15, IL 8, interferon alpha and CD 3.

In one embodiment, the antibodies are chosen from the group of antibodies comprising adalimumab, ABT874, etanercept, AMG714, HuMax-IL8, MEDI545, otelixizumab or infliximab.

In one embodiment, the antibodies are chosen from the group of antibodies or antibody fragments used in respiratory and pulmonary diseases, targeting:

IL 4, the IL 5 receptor, IL 1 (interleukin 1), IL 13, tumor necrosis factor receptor 1 (TNFR1), CD 25 (cluster of differentiation 25), CTGF (connective tissue growth factor), TNF alpha (tumor necrosis factor alpha), GM-CSF (granulocyte monocyte colony stimulating factor), CD 23, RSV (respiratory syncitial virus), IL 5, staphylococcus aureus clumping factor A, tissue factor, IgE (immunoglobulin E) or RSV (respiratory syncitial virus).

In one embodiment, the antibodies are chosen from the group of antibodies comprising AMG317, anti-IL13, BIW-8405, canakinumab, CAT354, CNTO148, daclizumab, FG-3019, GC-1008, golimumab, KB002, lumiliximab, MEDI557, mepolizumab, QAX576, tefibazumab, TNX-832, omalizumab or palivizumab.

The antibodies used in autoimmune and inflammatory diseases, chosen from antibodies or antibody fragments targeting:

TNF alpha (tumor necrosis factor alpha), CD 25 (cluster of differentiation 25), CD, LFA-1 (lymphocyte function-associated antigen), CD 3, IgE (immunoglobulin E), IL 6, B7RP-1 (B7-related protein), Blys (B lymphocyte stimulator), CCR4 (chemokine C—C receptor 4), CD 11a, CD 20 (cluster of differentiation 20), CD 22 (cluster of differentiation 22), CD 23, CD 4, CD 40, CD 44, CD 95, CXCL10, eotaxin 1, GM-CSF (granulocyte monocyte colony stimulating factor), IL 1 (interleukin 1), IL 12, IL 13, IL 15, IL 18, IL 5, IL 8, IL 23, integrin alpha 4 beta 7, integrins alpha 4 beta 1 or alpha 4 beta 7, interferon alpha, interferon gamma, interleukin-17 receptor, receptor activator of nuclear factor kappa B ligand (RANKL), VAP-1 (vascular adhesion protein-1) inflammation receptor or VAP-1 (vascular adhesion protein-1).

In one embodiment, the antibodies are chosen from the group of antibodies comprising adalimumab, basiliximab, daclizumab, efalizumab, muromonab-CD3, omalizumab or tocilizumab.

In one embodiment, the antibodies are chosen from the group of antibodies or antibody fragments used in cardiovascular and circulatory diseases, targeting:

glycoprotein IIb/IIIa receptor of human platelets, oxidized low-density lipoprotein (oxLDL), digoxin or factor VIII.

In one embodiment, the antibodies are chosen from the group of antibodies comprising abciximab, 7E3, BI-204, digibind or TB402.

In one embodiment, the antibodies are chosen from the group of antibodies or antibody fragments used in central nervous system diseases, targeting:

CD 52, integrins alpha 4 beta 1 or alpha 4 beta 7, beta amyloid peptide, IL 12, IL 23, CD 25 (cluster of differentiation 25), myelin-associated glycoprotein (MAG), CD 20 or NGF (neural growth factor).

In one embodiment, the antibodies are chosen from the group of antibodies comprising alemtuzumab, natalizumab, ABT874, Bapineuzumab, CNTO 1275, Daclizumab, GSK249320, rituximab or RN624.

In one embodiment, the antibodies are chosen from the group of antibodies or antibody fragments used in gastrointestinal diseases, targeting:

TNF alpha (tumor necrosis factor alpha), CD 25 (cluster of differentiation 25), toxin A of Clostridium difficile, CXCL10, IL 5 or integrins alpha 4 beta 1 or alpha 4 beta 7.

In one embodiment, the antibodies are chosen from the group of antibodies comprising infliximab, adalimumab, basiliximab, CNTO148, golimumab, MDX066, MDX1100, mepolizumab, MLN02 or Reslizumab.

The antibodies used in infectious diseases, chosen from antibodies or antibody fragments targeting:

hepatitis C virus sheath protein 2, PS (phosphatidyl serine), lipoteichoic acid, penicillin-binding protein (PBP), CD 4, CTLA4 (cytotoxic T-lymphocyte antigen 4, also known as: CD152), PD-1 (programmed death 1), West Nile virus, fungal antigen heat shock protein 90, CCR5 (chemokine C—C receptor 5), rabies virus, Bacillus anthracis protecting antigen, Staphylococcus aureus clumping factor A, Stx2 or TNF alpha (tumor necrosis factor alpha).

In one embodiment, the antibodies are chosen from the group of antibodies comprising bavituximab, peregrine, BSYXA110, cloxacillin, ibalizumab, MDX010, MDX1106, MGAWN1, Mycograb, Pro140, Rabies Antibody, raxibacumab, tefibazumab or TMA15.

In one embodiment, the antibodies are chosen from the group of antibodies or antibody fragments used in metabolic diseases and in endocrinology, targeting:

IL 1 (interleukin 1), GCGR (glucagon receptor), PTHrP (parathyroid hormone-related protein) or CD 3.

In one embodiment, the antibodies are chosen from the group of antibodies comprising IOR-T3, AMG108, AMG477, CAL, canakinumab, otelixizumab, teplizumab or XOMA052.

In one embodiment, the antibodies are chosen from the group of antibodies or antibody fragments used in female metabolic diseases, targeting:

the receptor activator of nuclear factor kappa B ligand (RANKL).

In one embodiment, the antibodies are chosen from the group of antibodies comprising Denosumab.

In one embodiment, the antibody is cetuximab.

In one embodiment, the antibody is bevacizumab.

The invention also relates to a process for optimizing the stabilization of a formulation of a monoclonal antibody, comprising the steps of:
providing a monoclonal antibody,
providing the library of amphiphilic polymers comprising the polysaccharides defined above,
measuring the thermal stabilization of said antibody,
determining the amphiphilic polysaccharide(s) capable of affording the best stabilization at the concentrations of the pharmaceutical formulations,
formulating said antibody in the presence of said amphiphilic polysaccharide(s).

In one embodiment, the measurement of the thermal stabilization is performed by incubating the antibody or the complex at 56° C. for 1 to 5 days. When the antibody alone or complexed is destabilized, it aggregates. This aggregation is monitored by measuring the light scattering at 450 nm.

The invention also relates to a pharmaceutical formulation comprising a composition according to the invention in which the polysaccharide/antibody mole ratio is between 0.2 and 20 and preferably between 0.5 and 10.

The antibody concentration in the formulations is preferably in the range between 1 mg/ml and about 250 mg/ml. This concentration is determined by the mode of formulation: for example, for an intravenous formulation, the concentration will be between 1 and 50 mg/ml, for a subcutaneous or intramuscular formulation, the concentration will be between 50 mg/ml and about 200 mg/ml.

The formulations are preferably aqueous formulations.

The formulations according to the invention may also comprise surfactants, for instance polysorbate, in concentrations of between 0.0001% and 1.0%.

The formulation may contain a salt or a nonionic species to maintain or restore the isotonicity, for example sodium chloride, glycerol or trehalose.

### EXAMPLE 1

### Synthesis of Sodium Dextran Methylcarboxylate Modified with Dodecylamine, Polymer 1

8 g (i.e. 148 mmol of hydroxyl functions) of dextran with a weight-average molar mass of about 40 kg/mol (Fluka) are dissolved in water to 42 g/L. 15 mL of 10 N NaOH (148 mmol of NaOH) are added to this solution. The mixture is brought to 35° C. and 23 g (198 mmol) of sodium chloroacetate are then added. The temperature of the reaction medium is maintained at 60° C. for 100 minutes. The reaction medium is diluted with 200 mL of water, neutralized with acetic acid and purified by ultrafiltration through 5 kD PES membrane against 6 volumes of water. The final solution is assayed by dry extract to determine the polymer concentration; and then assayed by acid/base titrimetry in 50/50 (V/V) water/acetone to determine the degree of substitution with methylcarboxylates.

According to the dry extract: [polymer]=31.5 mg/g.

According to the acid/base titration: the degree of substitution of the hydroxyl functions with methylcarboxylate functions is 1.04 per saccharide unit.

The sodium dextran methylcarboxylate solution is passed through a Purolite resin (anionic) to obtain dextran methylcarboxylic acid, which is then lyophilized for 18 hours.

7.5 g of dextran methylcarboxylic acid (34 mmol of methylcarboxylic acid functions) are dissolved in DMF to 45 g/L and then cooled to 0° C. 0.65 g of dodecylamine (3.5 mmol) and 3.69 g of triethylamine are dissolved in DMF to 100 g/L. Once the polymer solution is at 0° C., 3.69 g (36 mmol) of N-methylmorpholine and 4.98 g (36 mmol) of isobutylchloroformate are then added. After reaction for 10 minutes, the solution of dodecylamine and triethylamine is added. The medium is then maintained at 10° C. for 3 hours, and then heated to 20° C. Once at 20° C., 10 mL of water are added. The medium is poured into 820 mL of a 50/50 water/ethanol solution with vigorous stirring. The solution is ultrafiltered through a 5 kD PES membrane against 10 volumes of 0.9% NaCl solution and then 5 volumes of water. The concentration of the polymer solution is determined by dry extract. A fraction of the solution is lyophilized and analysed by 1H NMR in D₂O to determine the proportion of acid functions converted into dodecylamide.

According to the dry extract: [polymer 1]=25.9 mg/g

According to the 1H NMR: the mole fraction of acids modified with dodecylamine per saccharide unit is 0.10.

### EXAMPLE 2

### Synthesis of Sodium Dextran Methylcarboxylate Modified with Cholesteryl Leucinate, Polymer 2

Cholesteryl leucinate, para-toluenesulfonic acid salt, is obtained according to the process described in the patent (Kenji, M et al., U.S. Pat. No. 4,826,818).

The sodium dextran methylcarboxylate solution described in Example 1 is passed through a Purolite resin (anionic) to obtain dextran methylcarboxylic acid, which is then lyophilized for 18 hours.

8 g of dextran methylcarboxylic acid (37 mmol of methylcarboxylic acid functions) are dissolved in DMF to 45 g/L and then cooled to 0° C. 0.73 g of cholesteryl leucinate, para-toluenesulfonic acid salt (1 mmol) is suspended in DMF to 100 g/L. 0.11 g of triethylamine (1 mmol) is then added to this suspension. Once the polymer solution is at 0° C., 0.109 g (1 mmol) of NMM and 0.117 g (1 mmol) of EtOCOCl are added. After reaction for 10 minutes, the cholesteryl leucinate suspension is added. The medium is then maintained at 4° C. for 15 minutes. The medium is then heated to 30° C. Once at 30° C., the medium is poured into a solution of 3.76 g of NMM (37 mmol) at 5 g/L with vigorous stirring. The solution is ultrafiltered through a 10 kD PES membrane against 10 volumes of 0.9% NaCl solution and then 5 volumes of water. The concentration of the polymer solution is determined from the dry extract. A fraction of the solution is lyophilized and analysed by 1H NMR in D₂O to determine the proportion of acid functions converted into cholesteryl leucinate amide.

According to the dry extract: [polymer 2]=12.9 mg/g

According to the 1H NMR: the mole fraction of acids modified with cholesteryl leucinate per saccharide unit is 0.03.

### EXAMPLE 3

### Synthesis of Sodium Dextran Succinate Modified with Cholesteryl Leucinate, Polymer 3

Cholesteryl leucinate, para-toluenesulfonic acid salt, is obtained according to the process described in the patent (Kenji, M et al., U.S. Pat. No. 4,826,818).

Sodium dextran succinate is obtained from dextran 40 according to the method described in the article by Sanchez-Chaves et al. (Sanchez-Chaves, Manuel et al., Polymer 1998, 39 (13), 2751-2757.) The proportion of acid functions per glycoside unit (i) is 1.46 according to the 1H NMR in D₂O/NaOD.

The sodium dextran succinate solution is passed through a Purolite resin (anionic) to obtain dextran succinic acid, which is then lyophilized for 18 hours.

7.1 g of dextran succinic acid (23 mmol) are dissolved in DMF at 44 g/L. The solution is cooled to 0° C. 0.77 g of cholesteryl leucinate, para-toluenesulfonic acid salt (1 mmol) is suspended in DMF to 100 g/L. 0.12 g of triethylamine (TEA) (1 mmol) is then added to this suspension. Once the polymer solution is at 0° C., 0.116 g (1 mmol) of NMM and 0.124 g (1 mmol) of EtOCOCl are added. After reaction for 10 minutes, the cholesteryl leucinate suspension is added. The medium is then maintained at 4° C. for 15 minutes. The medium is then heated to 30° C. Once at 30° C., the medium is poured into a solution of 3.39 g of NMM (33 mmol) at 5 g/L with vigorous stirring. The solution is ultrafiltered through a 10 kD PES membrane against 10 volumes of 0.9% NaCl solution and then 5 volumes of water. The concentration of the polymer solution is determined from the dry extract. A fraction of the solution is lyophilized and analysed by 1H NMR in D₂O to determine the proportion of acid functions converted into cholesteryl leucinate amide.

According to the dry extract: [polymer 3]=17.5 mg/g

According to the 1H NMR: the mole fraction of acids modified with cholesteryl leucinate per saccharide unit is 0.05.

### EXAMPLE 4

### Synthesis of Sodium Dextran Methylcarboxylate Modified with Octyl Glycinate, Polymer 4

Octyl glycinate, para-toluenesulfonic acid salt, is obtained according to the process described in the patent (Kenji, M et al., U.S. Pat. No. 4,826,818).

Via a process similar to that described in Example 2, a sodium dextran methylcarboxylate modified with octyl glycinate is obtained.

According to the dry extract: [polymer 4]=34.1 mg/g

According to the 1H NMR: the mole fraction of acids modified with octyl glycinate per saccharide unit is 0.1.

### EXAMPLE 5

### Synthesis of Sodium Dextran Methylcarboxylate Modified with Isohexyl Leucinate, Polymer 5

Isohexyl leucinate, para-toluenesulfonic acid salt, is obtained according to the process described in the patent (Kenji, M et al., U.S. Pat. No. 4,826,818).

According to a process similar to that described in Example 2, a sodium dextran methylcarboxylate modified with isohexyl leucinate is obtained.

According to the dry extract: [polymer 5]=16 mg/g

According to the 1H NMR: the mole fraction of acids modified with isohexyl leucinate per saccharide unit is 0.17.

### EXAMPLE 6

### Synthesis of Sodium Dextran Methylcarboxylate Modified with Dodecyl Phenylalaninate, Polymer 6

Dodecyl phenylalaninate, para-toluenesulfonic acid salt, is obtained according to the process described in the patent (Kenji, M et al., U.S. Pat. No. 4,826,818).

Via a process similar to that described in Example 2, a sodium dextran methylcarboxylate modified with dodecyl phenylalaninate is obtained.

According to the dry extract: [polymer 6]=20 mg/g

According to the 1H NMR: the mole fraction of acids modified with dodecyl phenylalaninate per saccharide unit is 0.1.

### EXAMPLE 7

### Synthesis of Sodium Dextran Methylcarboxylate Modified with Benzyl Phenylalaninate, Polymer 7

According to a process similar to that described in Example 2, a sodium dextran methylcarboxylate modified with benzyl phenylalaninate is obtained by using benzyl phenylalaninate, hydrogen chloride salt (Bachem).

According to the dry extract: [polymer 7]=47.7 mg/g

According to the 1H NMR: the mole fraction of acids modified with benzyl phenylalaninate per saccharide unit is 0.41.

### EXAMPLE 8

### Synthesis of Sodium Dextran Methylcarboxylate Modified with Dodecyl Glycinate, Polymer 8

Dodecyl glycinate, para-toluenesulfonic acid salt, is obtained according to the process described in the patent (Kenji, M et al., U.S. Pat. No. 4,826,818).

According to a process similar to that described in Example 2, a sodium dextran methylcarboxylate modified with dodecyl glycinate is obtained.

According to the dry extract: [polymer 8]=25.3 mg/g

According to the 1H NMR: the mole fraction of acids modified with dodecyl glycinate per saccharide unit is 0.1.

### EXAMPLE 9

### Synthesis of Sodium Dextran Methylcarboxylate Modified with Decyl Glycinate, Polymer 9

Decyl glycinate, para-toluenesulfonic acid salt, is obtained according to the process described in the patent (Kenji, M et al., U.S. Pat. No. 4,826,818).

According to a process similar to that described in Example 2, a sodium dextran methylcarboxylate modified with decyl glycinate is obtained.

According to the dry extract: [polymer 9]=23.1 mg/g

According to the 1H NMR: the mole fraction of acids modified with dodecyl glycinate per saccharide unit is 0.1.

### EXAMPLE 10

### Synthesis of Sodium Dextran Methylcarboxylate Modified with Octanol, Polymer 10

1-Octyl p-toluenesulfonate is obtained according to the process described in the publication (Morita, J.-I. et al., Green Chem. 2005, 7, 711).

Sodium dextran methylcarboxylate is synthesized according to the process described in Example 1, using a dextran with a weight-average molecular mass of about 10 kg/mol (Pharmacosmos).

The sodium dextran methylcarboxylate solution is passed through a Purolite resin (anionic) to obtain an aqueous solution of dextran methylcarboxylic acid whose pH is raised to 7.1 by adding aqueous (40%) tetrabutylammonium hydroxide (Sigma) solution, and the solution is then lyophilized for 18 hours.

20 g of tetrabutylammonium dextran methylcarboxylate (45 mmol of methylcarboxylate functions) are dissolved in DMF to 120 g/L and then heated to 40° C. A solution of 2.37 g of 1-octyl p-toluenesulfonate (8.3 mmol) in 12 mL of DMF is then added to the polymer solution. The medium is then maintained at 40° C. for 5 hours. The solution is ultrafiltered through a 10 kD PES membrane against 15 volumes of 0.9% NaCl solution and then 5 volumes of water. The concentration of the polymer solution is determined from the dry extract. A fraction of the solution is lyophilized and analysed by 1H NMR in D₂O to determine the proportion of acid functions converted into the 1-octyl ester.

According to the dry extract: [polymer 10]=20.2 mg/g

According to the 1H NMR: the mole fraction of acids modified with 1-octanol per saccharide unit is 0.17.

### EXAMPLE 11

### Synthesis of Sodium Dextran Methylcarboxylate Modified with Dodecanol, Polymer 11

1-Dodecyl p-toluenesulfonate is obtained according to the process described in the publication (Morita, J.-I. et al., Green Chem. 2005, 7, 711).

Via a process similar to that described in Example 10, using a dextran with a weight-average molecular mass of about 10 kg/mol (Pharmacosmos), a sodium dextran methylcarboxylate modified with dodecanol is obtained.

According to the dry extract: [polymer 11]=18.7 mg/g

According to the 1H NMR: the mole fraction of acids modified with dodecanol per saccharide unit is 0.095.

### EXAMPLE 12

### Synthesis of Sodium Dextran Methylcarboxylate modified with phenylalaminol caprylate ester, Polymer 13

Phenylalaminol caprylate ester, para-toluenesulfonic acid salt, is obtained according to the process described in the patent (Kenji, M et al., U.S. Pat. No. 4,826,18).

Via a process similar to that described in Example 2, a sodium dextran methylcarboxylate modified with phenylalaminol caprylate ester is obtained.

According to the dry extract: [polymer 13]=25 mg/g

According to the 1H NMR: the mole fraction of acids modified with phenylalaminol caprylate ester per saccharide unit is 0.045.

### EXAMPLE 13

### Synthesis of Sodium Dextran Methylcarboxylate Modified with Ethanolamine Caprylate Ester, Polymer 14

Ethanolamine caprylate ester, para-toluenesulfonic acid salt, is obtained according to the process described in the patent (Kenji, M et al., U.S. Pat. No. 4,826,818).

Via a process similar to that described in Example 2, a sodium dextran methylcarboxylate modified with ethanolamine caprylate ester is obtained.

According to the dry extract: [polymer 14]=29.1 mg/g

According to the 1H NMR: the mole fraction of acids modified with ethanolamine caprylate ester per saccharide unit is 0.15.

### EXAMPLE 14

### Synthesis of Sodium Dextran Methylcarboxylate Modified with Ethanolamine Laurate Ester, Polymer 15

Ethanolamine laurate ester, para-toluenesulfonic acid salt, is obtained according to the process described in the patent (Kenji, M et al., U.S. Pat. No. 4,826,818).

According to a process similar to that described in Example 2, a sodium dextran methylcarboxylate modified with ethanolamine laurate ester is obtained.

According to the dry extract: [polymer 15]=21.2 mg/g

According to the 1H NMR: the mole fraction of acids modified with ethanolamine laurate ester per saccharide unit is 0.09.

### EXAMPLE 15

### Counterexample 1, Synthesis of Dextran Methylcarboxylate not Modified with a Hydrophobic Group, Polymer 16

Sodium dextran methylcarboxylate is obtained as described in the first part of Example 1. The mole fraction of acids modified by a hydrophobic group is zero.

### EXAMPLE 16

### Thermal Stabilization of Antibodies by Complexing with the Polymers

Description of the Stability Test

This test makes it possible to measure the thermal stabilization of monoclonal antibodies by interaction with polymers. The thermal stability takes place by incubating the antibody or the complex at 56° C. for 1 to 5 days. When the antibody alone or in complexed form is destabilized, it aggregates. This aggregation is monitored by measuring the light scattering at 450 nm.

Determination of the Test Concentration of Antibodies

Despite their similarity, monoclonal antibodies have different solubilities or stabilities at the formulation concentrations. To use this test, an antibody concentration that makes it possible to measure a sufficient destabilization signal must first be determined. To do this, 200 μl of monoclonal antibody at concentrations of 1, 2, 4, 6 and 10 mg/ml, for example, is incubated at 56° C. for 48 hours. The absorbance at 450 nm is measured at t0 and at t48h. The test concentration is determined as the minimum concentration for which the difference in absorbance between t48h and t0 is at least 0.5 for an optical path length of 1 cm.

Study of the Polymer-Mediated Stabilization

100 μl of antibodies at twice the test concentration are mixed with 100 μl of polymer at the same molar concentration so as to obtain an antibody solution at the test concentration in the presence of polymer in a 1/1 mole ratio. The formulation is incubated at 56° C. for 5 days and the absorbance at 450 nm is measured at t0, t24h, t48h and t96h and then every 24 hours. A polymer is considered to be positive (+) if it leads to a lower absorbance than that obtained with the antibody alone at the various analysis times. A polymer is considered to be very positive (++) if it leads to a much lower absorbance than that obtained with the antibody alone at the various analysis times. In both cases, this indicates lower aggregation of the monoclonal antibody and thus thermal stabilization of the monoclonal antibody by the polymer. The polymer is considered to be negative (−) if it leads to an absorbance that is substantially identical to that obtained with the antibody alone at the various analysis times.

Results obtained:

Cetuximab (Erbitux) at 1.3 mg/ml

| Polymer | Stabilization |
|---|---|
| 1 | ++ |
| 2 | ++ |
| 3 | ++ |
| 4 | + |
| 5 | + |
| 6 | + |
| 7 | + |
| 8 | ++ |
| 9 | + |
| 16 | − |

Bevacizumab (Avastin) at 6 mg/ml

| Polymer | Stabilization |
|---|---|
| 1 | ++ |
| 2 | ++ |
| 4 | − |
| 8 | ++ |
| 9 | + |
| 10 | + |
| 16 | − |

### EXAMPLE 17

### Study of the Effect of the Carbon Chain Length of the Graft on the Stabilization

Polymers 4, 9 and 8 differ by the length of their fatty chain, ranging from C8 to C12. Their stabilizing effect described in Example 17 is summarized in the following table.

| | | Stabilization of | Stabilization of |
|---|---|---|---|
| | Fatty chain | Cetuximab | Bevacuzimab |
| Polymer | length | at 1.3 mg/ml | at 10 mg/ml |
| 4 | C8 | + | − |
| 9 | C10 | + | + |
| 8 | C12 | ++ | ++ |

The results obtained clearly show that increasing the fatty chain length induces better stabilization.

### EXAMPLE 18

### Study of the Polymer-Mediated Stabilization as a Function of the Ionic Strength

6.4 mL of Avastin at 25 mg/ml and 50 mM phosphate pH 6.2 are mixed with 0.165 mL of 4 M NaCl and 1.435 mL of 50 mM phosphate to obtain Avastin at 20 mg/ml in 50 mM phosphate, 83 mM NaCl. 2.5 ml of Polymer 8 at 11 mg/ml in 50 mM phosphate pH 6.2, 83 mM NaCl are added to 2.5 ml of this Avastin solution, to give a polymer/Avastin complex solution in a 2/1 mole ratio containing 10 mg/ml of Avastin. An identical solution is prepared without polymer.

2 ml of each solution are stored for the stabilization study at high ionic strength and 3 ml are diafiltered to obtain a sample of low ionic strength: 3 ml of Avastin/Polymer complex or of Avastin solution alone are diluted four-fold by adding 9 ml of H₂O and then centrifuged in an amicon equipped with a 10 kD membrane, until a volume of 3 ml is obtained. This step is repeated twice with 5 mM phosphate pH 6.2 buffer.

The four formulations are incubated at 56° C. for 4 hours, and the absorbance at 450 nm is measured at T0, T60h and T80h. A reduction in the increase of absorbance over time relative to that of the antibody alone indicates lower aggregation and thus thermal stabilization of the antibody.

| | High ionic strength | Low ionic strength |
|---|---|---|
| | (150 mM) | (7 mM) |
| Avastin alone | −− | − |
| 10 mg/ml | | |
| Avastin | + | ++ |
| 10 mg/ml + Polymer 8 | | |

Under these conditions, the formulations containing complex are more stable than those containing antibody alone. Furthermore, the stability increases as the ionic strength decreases.

### EXAMPLE 19

### Stabilization of a Monoclonal Antibody with Respect to Mechanical Stress

The monoclonal antibody Avastin is diluted to 2 mg/mL from a stock solution at 25 mg/mL and 50 mM phosphate, pH 6.2 (a first dilution is made to 1/5 with purified water and the next one to 2/5 with 10 mM phosphate buffer). The final phosphate concentration is 10 mM. A polymer solution is prepared from lyophilizate in a 10 mM, pH 6.2 phosphate buffer such that volume-for-volume mixing with the previous solution makes it possible to obtain the monoclonal antibody at 1 mg/mL, 10 mM of phosphate and a polymer/antibody mole ratio of 3. The formulations are then filtered through a filter of 0.22 μm porosity and distributed into transparent 2 mL HPLC flasks.

The samples are then exposed to a mechanical stress using a magnetic bar with a glass surface, at a speed of 130 rpm. Samples are taken at various intervals and analysed by dynamic light scattering in order to determine the state of aggregation of the antibody.

A sample is designated as “+” if the aggregation is moderately inhibited by the polymer present. A sample is designated as “++” if the aggregation is more strongly inhibited. A sample is designated as “+++” if the aggregation is very strongly inhibited by the polymer present.

The results are given in the table below:

| Solution | Stability |
|---|---|
| Avastin alone | − |
| Polymer 16 | + |
| Polymer 13 | + |
| Polymer 15 | ++ |
| Polymer 14 | ++ |
| Polymer 8 | +++ |

The effect of the polymer not modified with a hydrophobe, Polymer 16, on the aggregation of the antibody induced by the mechanical stress is low. On the other hand, the polymers modified with a hydrophobe have a greater effect on inhibition of the aggregation, which may go as far as stabilizing the Polymer 8.

1. A stable pharmaceutical composition comprising at least one monoclonal antibody and at least one amphiphilic polysaccharide.

2. The composition as claimed in claim 1, wherein the amphiphilic polysaccharide is chosen from the group of amphiphilic polysaccharides comprising carboxyl functional groups partly substituted with at least one hydrophobic substituent.

3. The composition as claimed in claim 1, wherein the amphiphilic polysaccharide is chosen from polysaccharides comprising carboxyl functional groups, at least one of which is substituted with a hydrophobic radical, noted Hy:
said hydrophobic radical (Hy) being grafted or bound to the anionic polysaccharide either:
via a function F′, said function F′ resulting from coupling between a reactive function of a hydrophobic compound and a carboxyl function of the anionic polysaccharide,
via a linker R, said linker R being linked to the polysaccharide via a bond F resulting from coupling between a reactive function of the precursor of the linker R′ and a carboxyl function of the anionic polysaccharide and said hydrophobic radical (Hy) being linked to the linker R via a function G resulting from coupling between a reactive function of a hydrophobic compound and a reactive function of the precursor of the linker R′;
the carboxyl functions of the unsubstituted anionic polysaccharide being in the form of the carboxylate of a cation,
F being either an amide, ester, thioester or anhydride function,
F′ being either an amide, ester, thioester or anhydride function,
G being either an amide, ester, thioester, thionoester, carbamate, carbonate or anhydride function,
Hy being a radical resulting either from coupling between a reactive function of a hydrophobic compound and a carboxyl function of the anionic polysaccharide, or from coupling between a reactive function of a hydrophobic compound and a reactive function of the precursor of the linker R′, consisting of a chain comprising between 4 and 50 carbons, optionally branched and/or unsaturated, optionally comprising one or more heteroatoms, such as O, N and/or S, optionally comprising one or more saturated, unsaturated or aromatic rings or heterocycles,
R being a divalent radical consisting of a chain comprising between 1 and 18 carbons, optionally branched and/or unsaturated, optionally comprising one or more heteroatoms, such as O, N and/or S, optionally comprising one or more saturated, unsaturated or aromatic rings or heterocycles and resulting from the reaction of a precursor R′ containing at least two identical or different reactive functions chosen from the group consisting of alcohol, acid, amine, thiol and thio acid functions,
said polysaccharide comprising carboxyl functional groups being amphiphilic at neutral pH.

4. The composition as claimed in claim 1, wherein the amphiphilic polysaccharides are chosen from polysaccharides comprising carboxyl functional groups, at least one of which is substituted with a hydrophobic alcohol derivative, noted Ah:
said hydrophobic alcohol (Ah) being grafted or linked to the anionic polysaccharide via a coupling arm R, said coupling arm being linked to the anionic polysaccharide via a function F, said function F resulting from coupling between an amine, alcohol, thioalcohol or carboxyl function of the precursor of the linker R′ and a carboxyl function of the anionic polysaccharide, and said coupling arm R being linked to the hydrophobic alcohol via a function G resulting from coupling between a carboxyl, amine, thio acid or alcohol function of the precursor of the coupling arm R′ and an alcohol function of the hydrophobic alcohol, the carboxyl functions of the unsubstituted anionic polysaccharide being in the form of a carboxylate of a cation,
F being either an amide function or an ester function, or a thioester function, or an anhydride function,
G being either an ester function, or a thioester function, or a carbonate function, or a carbamate function,
R being a divalent radical consisting of a chain comprising between 1 and 18 carbons, optionally branched and/or unsaturated, optionally comprising one or more heteroatoms, such as O, N and/or S,
Ah being a hydrophobic alcohol or thioalcohol residue, produced from coupling between the hydroxyl function of the hydrophobic alcohol and at least one reactive function borne by the precursor of the divalent radical R,
said polysaccharide comprising carboxyl functional groups being amphiphilic at neutral pH.

5. The composition as claimed in claim 1, wherein the amphiphilic polysaccharides are chosen from polysaccharides comprising carboxyl functional groups, at least one of which is substituted with a hydrophobic alcohol derivative, noted Ah:
said hydrophobic alcohol (Ah) being grafted or linked to the anionic polysaccharide via a function F′, said function F′ resulting from coupling between the carboxylate function of the anionic polysaccharide and the hydroxyl function of the hydrophobic alcohol, the unsubstituted carboxyl functions of the anionic polysaccharide being in the form of the carboxylate of a cation,
F′ being an ester or thioester function,
Ah being a hydrophobic alcohol residue or a hydrophobic thioalcohol residue,
said polysaccharide comprising carboxyl functional groups being amphiphilic at neutral pH.

6. The composition as claimed in claim 1, wherein the amphiphilic polysaccharide is chosen from the group of polysaccharides comprising carboxyl functional groups, at least one of which is substituted with a hydrophobic amine derivative, noted Amh:
said hydrophobic amine being grafted or linked to the anionic polysaccharide via an amide function F′, said amide function F′ resulting from coupling between the amine function of the hydrophobic amine and a carboxyl function of the anionic polysaccharide, the unsubstituted carboxyl functions of the anionic polysaccharide being in the form of a carboxylate of a cation,
Amh being a hydrophobic amine residue produced by coupling between the amine function of the hydrophobic amine and a carboxyl function of the anionic polysaccharide.

7. The composition as claimed in claim 1, wherein the amphiphilic polysaccharide is chosen from the group of polysaccharides comprising carboxyl functional groups, at least one of which is substituted with a hydrophobic acid derivative, noted Ach:
said hydrophobic acid (Ach) being grafted or linked to the anionic polysaccharide via an anhydride function F′, said function F′ resulting from coupling between the carboxyl function of the anionic polysaccharide and the carboxyl function of the hydrophobic acid, the unsubstituted carboxyl functions of the anionic polysaccharide being in the form of the carboxylate of a cation,
Ach being a hydrophobic acid or hydrophobic O-thioacid residue,
said polysaccharide comprising carboxyl functional groups being amphiphilic at neutral pH.

8. The composition as claimed in claim 1, wherein the amphiphilic polysaccharide is chosen from the group of polysaccharides comprising carboxyl functional groups, at least one of which is substituted with a hydrophobic acid derivative, noted Ach:
said hydrophobic acid (Ach) being grafted or linked to the anionic polysaccharide via a coupling arm R, said coupling arm being linked to the anionic polysaccharide via a function F, said function F resulting from coupling between an amine, alcohol, thioalcohol or carboxyl function of the precursor of the linker R′ and a carboxyl function of the anionic polysaccharide, and said coupling arm R being linked to the hydrophobic acid via a function G resulting from coupling between an amine, alcohol, thioalcohol or carboxyl function of the precursor of the coupling arm R′ and a carboxyl function of the hydrophobic acid, the unsubstituted carboxyl functions of the anionic polysaccharide being in the form of the carboxylate of a cation,
F being either an amide function, or an ester function, or a thioester function, or an anhydride function,
G being either an ester function, or an amide function, or a thioester function, or an anhydride function,
R being a divalent radical consisting of a chain comprising between 1 and 18 carbons, optionally branched and/or unsaturated, optionally comprising one or more heteroatoms such as O, N and/or S,
Ach being a residue of an acid, produced by coupling between the carboxyl function of the hydrophobic acid and at least one reactive function borne by the precursor R′ of the divalent radical R,
said polysaccharide comprising carboxyl functional groups being amphiphilic at neutral pH.

9. The composition as claimed in claim 1, wherein the polysaccharides comprising carboxyl functional groups are polysaccharides naturally bearing carboxyl functional groups and are chosen from the group consisting of alginate, hyaluronan and galacturonan.

10. The composition as claimed in claim 1, wherein the polysaccharides comprising carboxyl functional groups are synthetic polysaccharides obtained from polysaccharides naturally comprising carboxyl functional groups or from neutral polysaccharides on which at least 15 carboxyl functional groups per 100 saccharide units have been grafted, of general formula II: 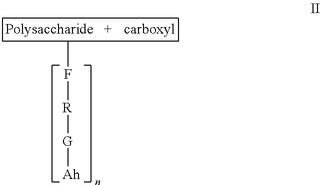
the natural polysaccharides being chosen from the group of polysaccharides predominantly consisting of glycoside bonds of (1,6) and/or (1,4) and/or (1,3) and/or (1,2) type,
L being a bond resulting from coupling between the linker Q and an —OH function of the polysaccharide and being either an ester, thioester, carbonate, carbamate or ether function,
i represents the mole fraction of the substituents L-Q per saccharide unit of the polysaccharide,
Q being a chain comprising between 1 and 18 carbons, optionally branched and/or unsaturated, comprising one or more heteroatoms, such as O, N and/or S, and comprising at least one carboxyl functional group, —CO₂H.

11. The composition as claimed in claim 1, wherein the polysaccharide is consisted predominantly of glycoside bonds of (1,6) type and is dextran.

12. The composition as claimed in claim 1, wherein the polysaccharide is consisted predominantly of glycoside bonds of (1,4) type and is chosen from the group consisting of pullulan, alginate, hyaluronan, xylan, galacturonan or a water-soluble cellulose.

13. The composition as claimed in claim 1, wherein the polysaccharide is consisted predominantly of glycoside bonds of (1,3) type and is a curdlan.

14. The composition as claimed in claim 1, wherein the polysaccharide is consisted predominantly of glycoside bonds of (1,2) type and is an inulin.

15. The composition as claimed in claim 1, wherein the polysaccharide is consisted predominantly of glycoside bonds of (1,4) and (1,3) type and is a glucan.

16. The composition as claimed in claim 1, wherein the polysaccharide is consisted predominantly of glycoside bonds of (1,4) and (1,3) and (1,2) type and is mannan.

17. The composition as claimed in claim 1, wherein the antibody is chosen from the group of antibodies or antibody fragments used in cancerology, targeting:
CD 52, VEGF (vascular endothelial growth factor), EGF R (epidermal growth factor receptor), CD11a, CCR4 (chemokine C—C receptor 4), CD 105, CD 123, CD 137, CD 19, CD 22, CD 23, CD 3, CD 30, CD 38, CD 4, CD 40, CD 55SC-1, CD 56, CD 6, CD 74, CD 80, CS 1 (cell-surface glycoprotein 1), CTLA4 (cytotoxic T-lymphocyte antigen 4, also known as CD152), DR5 (death receptor 5), Ep-CAM (epithelial cell adhesion molecule), folate receptor alpha, ganglioside GD2, ganglioside GD3, GPNMB, glycoprotein NMB, HGF/SF (hepatocyte growth factor/scatter factor), IGF-1 (insulin-like growth factor), IGF1-receptor (insulin-like growth factor-1 receptor), IL 13 (interleukin-13), IL 6 (interleukin-6), IL-6R (interleukin-6 receptor), immunodominant fungal antigen heat shock protein 90 (hsp90), integrin alpha 5 beta 3, MHC (major histocompatibility complex) class II, MN-antigen (also known as G250 antigen), MUC1, PD-1 (programmed death 1), PIGF (placental growth factor), PDGFRa (platelet-derived growth factor receptor alpha), prostate specific membrane antigen (PSMA), PTHrP (parathyroid hormone-related protein), CD200 receptor, receptor activator of nuclear factor kappa B ligand (RANKL), sphingosine-1-phosphate (SIP), TGF beta (transforming growth factor beta), TRAIL (tumor necrosis factor (TNF)-related apoptosis-inducing ligand) receptor 1, tumor necrosis factor receptor 2, vascular endothelial growth factor receptor 2 (VEGFR-2), CD 33, CD 20, CA125 (cancer antigen 125) or epidermal growth factor receptor.

18. The composition as claimed in claim 17; wherein the antibody is chosen from the group of antibodies comprising alemtuzumab, bevacizumab, cetuximab, efalizumab, gemtuzumab, britumomab, ovarex mab, panitumumab, rituximab, tositumomab and trastuzumab.

19. The composition as claimed in claim 1, wherein the antibody is chosen from the group of antibodies or antibody fragments used in dermatology, targeting:
TNF alpha (tumor necrosis factor alpha), IL 12, IL 15, IL 8, interferon alpha and CD 3.

20. The composition as claimed in claim 19, wherein the antibody is chosen from the group of antibodies comprising adalimumab, ABT874, etanercept, AMG714, HuMax-IL8, MEDI545, otelixizumab and infliximab.

21. The composition as claimed in claim 1, wherein the antibody is chosen from the group of antibodies or antibody fragments used in respiratory and pulmonary diseases, targeting:
IL 4 and 13, the IL 5 receptor, IL 1 (interleukin 1), tumor necrosis factor receptor 1 (TNFR1), CD 25 (cluster of differentiation 25), CTGF (connective tissue growth factor), TNF alpha (tumor necrosis factor alpha), GM CSF (granulocyte monocyte colony stimulating factor), CD 23, RSV (respiratory syncitial virus), IL 5, staphylococcus aureus clumping factor A, or tissue factor, IgE (immunoglobulin E).

22. The composition as claimed in claim 21, wherein the antibody is chosen from the group of antibodies comprising AMG317, anti-IL13, BIW-8405, canakinumab, CAT354, CNTO148, daclizumab, FG-3019, GC 1008, golimumab, KB002, lumiliximab, MEDI557, mepolizumab, QAX576, tefibazumab, TNX-832, omalizumab and palivizumab.

23. The composition as claimed in claim 1, wherein the antibody is chosen from the group of antibodies or antibody fragments used in autoimmune and inflammatory diseases, chosen from antibodies targeting:
TNF alpha (tumor necrosis factor alpha), CD 25 (cluster of differentiation 25), CD, LFA-1 (lymphocyte function-associated antigen), CD 3, IgE (immunoglobulin E), IL 6, B7RP-1 (B7-related protein), Blys (B lymphocyte stimulator), CCR4 (chemokine C—C receptor 4), CD11a, CD 20 (cluster of differentiation 20), CD 22 (cluster of differentiation 22), CD 23, CD 4, CD 40, CD 44, CD 95, CXCL10, eotaxin 1, GM-CSF (granulocyte monocyte colony stimulating factor), IL 1 (interleukin 1), IL 12, IL 13, IL 15, IL 18, IL 5, IL 8, IL 23, integrin alpha 4 beta 7, integrins alpha 4 beta 1 or alpha 4 beta 7, interferon alpha, interferon gamma, interleukin-17 receptor, receptor activator of nuclear factor kappa B ligand (RANKL), VAP-1 (vascular adhesion protein-1) inflammation receptor or VAP-1 (vascular adhesion protein-1).

24. The composition as claimed in claim 23, wherein the antibody is chosen from the group of antibodies comprising adalimumab, basiliximab, daclizumab, efalizumab, muromonab-CD3, omalizumab and tocilizumab.

25. The composition as claimed in claim 1, wherein the antibody is chosen from the group of antibodies or antibody fragments used in cardiovascular and circulatory diseases, targeting:
glycoprotein IIb/IIIa receptor of human platelets, oxidized low-density lipoprotein (oxLDL), digoxin or factor VIII.

26. The composition as claimed in claim 24, wherein the antibody is chosen from the group of antibodies comprising abciximab, 7E3, BI-204, Digibind and TB402.

27. The composition as claimed in claim 1, wherein the antibody is chosen from the group of antibodies or antibody fragments used in central nervous system diseases, targeting:
CD 52, integrins alpha 4 beta 1 or alpha 4 beta 7, beta amyloid peptide, IL 12, IL 23, CD 25 (cluster of differentiation 25), myelin-associated glycoprotein (MAG), CD 20 or NGF (neural growth factor).

28. The composition according to claim 27, wherein the antibody is chosen from the group of antibodies comprising alemtuzumab, natalizumab, ABT874, Bapineuzumab, CNTO 1275, Daclizumab, GSK249320, rituximab and RN624.

29. The composition as claimed in claim 25, wherein the antibody is chosen from the group of antibodies or antibody fragments used in gastrointestinal diseases, targeting:
TNF alpha (tumor necrosis factor alpha), CD 25 (cluster of differentiation 25), toxin A of Clostridium difficile, CXCL10, IL 5 or integrins alpha 4 beta 1 or alpha 4 beta 7.

30. The composition as claimed in claim 29, wherein the antibody is chosen from the group of antibodies comprising infliximab, adalimumab, basiliximab, CNTO148, golimumab, MDX066, MDX1100, mepolizumab, MLN02 and Reslizumab.

31. The composition as claimed in claim 1, wherein the antibody is chosen from the group of antibodies or antibody fragments used in infectious diseases, chosen from antibodies targeting:
hepatitis C virus sheath protein 2, PS (phosphatidyl serine), lipoteichoic acid, penicillin-binding protein (PBP), CD 4, CTLA4 (cytotoxic T-lymphocyte antigen 4, also known as: CD152), PD-1 (programmed death 1), West Nile virus, fungal antigen heat shock protein 90, CCR5 (chemokine C—C receptor 5), rabies virus, Bacillus anthracis protecting antigen, Staphylococcus aureus clumping factor A, Stx2 or TNF alpha (tumor necrosis factor alpha).

32. The composition as claimed in claim 31, wherein the antibody is chosen from the group of antibodies comprising Bavituximab, Peregrine, BSYXA110, cloxacillin, ibalizumab, MDX010, MDX1106, MGAWN1, Mycograb, Pro140, Rabies Antibody, raxibacumab, tefibazumab and TMA15.

33. The composition as claimed in claim 1, wherein the antibody is chosen from the group of antibodies or antibody fragments used in metabolic diseases and in endocrinology, targeting:
CD 3, IL 1 (interleukin 1), GCGR (glucagon receptor), or PTHrP (parathyroid hormone-related protein).

34. The composition as claimed in claim 31, wherein the antibody is chosen from the group of antibodies comprising IOR-T3, AMG108, AMG477, CAL, canakinumab, otelixizumab, Teplizumab and XOMA052.

35. The composition as claimed in claim 1, wherein the antibody is chosen from the group of antibodies used in female metabolic diseases, targeting:
receptor activator of nuclear factor kappa B ligand (RANKL).

36. The composition as claimed in claim 35, wherein that the antibody is chosen from the group of antibodies comprising Denosumab.

37. The composition as claimed in claim 1, wherein the antibody is bevacizumab.

38. The composition as claimed in claim 1, wherein the antibody is cetuximab.

39. A pharmaceutical composition comprising a composition as claimed in claim 1, in which the polysaccharide/antibody mole ratio is between 0.2 and 20.

40. A process for optimizing the stabilization of a formulation of a monoclonal antibody, comprising the steps of:
providing a monoclonal antibody,
providing the library of amphiphilic polymers comprising the polysaccharides defined above,
measuring the thermal stabilization of said antibody,
determining the amphiphilic polysaccharide(s) capable of affording the best stabilization at the concentrations of the pharmaceutical formulations,
formulating said antibody in the presence of said amphiphilic polysaccharide(s).
